(19) United States
(12) Patent Application Publication
(10) Pub. No.: US 2025/0369989 A1
(43) Pub. Date: Dec. 4, 2025

(54) METHODS FOR DETECTING B-ISOX PRECIPITATES OR CAPTURED PROTEINS AS BIOFLUID BIOMARKERS

(71) Applicant: YEEFAN MED INC, Temple City, CA (US)

(72) Inventors: I-Fan WANG, Caotun Township (TW); Hsiang-Yu CHANG, Taichung City (TW); Chen-Hung TING, Yilan City (TW)

(21) Appl. No.: 18/874,670

(22) PCT Filed: Jun. 12, 2023
(86) PCT No.: PCT/US2023/025073

§ 371 Date: Dec. 13, 2024

Related U.S. Application Data
(60) Provisional application No. 63/351,813, filed on Jun. 13, 2022.

(51) Int. Cl. G01N 33/68; G01N 33/543

(52) U.S. Cl. CPC G01N 33/6896; G01N 33/54366; G01N 2333/4703; G01N 2474/10; G01N 2800/2821; G01N 2800/2835

(57) ABSTRACT

Described herein are detecting methods for conformational disease, aging and proteinopathies, by measuring the presence of b-isox-precipitates and the levels of b-isox-captured proteins in biofluids of healthy individuals and patients. Research identified additional biomarkers, which made it possible to detect, diagnose or treat, a human disease in a human subject by, with or without adding an isoxazole to an obtained biofluid sample, detecting the biomarker. Use of b-iso and/or biomarkers for diagnosing the disease are made possible.

### CROSS REFERENCE

The present application claims priority benefit of U.S. Provisional Application No. 63/351,813, filed 13 Jun. 2022, the entire contents of which application are hereby incorporated herein by reference.

### TECHNOLOGY FIELD

A method detects, and makes it possible to diagnose or treat, a human disease in a human subject by adding an isoxazole to an obtained biofluid sample to form a biofluid isoxazole composition in the biofluid sample, and detecting a presence of the biofluid isoxazole composition. Research identified additional biomarkers, which made it possible to detect, diagnose or treat, a human disease in a human subject by, with or without adding an isoxazole to an obtained biofluid sample, detecting the biomarker.

Although usable for other purposes, the methods relate to methods for differential diagnostics, real-time pathophysiology monitoring, presymptomatic diagnostics, and pharmacoresponse measurement of conformational diseases and proteinopathies, such as neurodegenerative diseases, diabetes, cancer, psychiatric disorders, and even aging.

### BACKGROUND OF THE INVENTION

Conformational diseases include more than 50 disorders caused by the accumulation of unfolded or misfolded proteins. Improper protein folding can lead to deposit amorphous aggregates, such as p-TDP-43 aggregates, or ordered amyloid fibrils, such as synuclein and tau inclusions (1).

Proteinopathies refer to pathologies caused by certain misfolded aggregation-prone proteins, such as synuclein, TDP-43 and tau. For example, synucleinopathies and tauopathy.

Amyotrophic lateral sclerosis (ALS) is a progressive, heterogeneous neuromuscular disease with limited treatment options (2). ALS degenerative symptoms are characterized by adult-onset progressive loss of primarily motor neurons in the cerebral cortex, brain stem, and spinal cord, consequently leading to progressive muscle weakness, atrophy and typical death 3 to 5 years after symptom onset. Although few ALS-causing gene, including SOD1, TDP-43and C9orf72, have been discovered for ALS and their proteins convert to misfold structure and form pathological inclusions, current treatments or diagnostics are still hampered by lack of definitive targets (3-7). Currently, the typical time to ALS diagnosis is 10-16 months from symptom onset. Misdiagnoses are a key factor leading to diagnostic delay. So far, no biomarker is available for sporadic ALS, which covers 90% patients with ALS.

Alzheimer's disease (AD) is the most common cause of memory loss and cognitive abilities (e.g., dementia) and may contribute to 60-70% of cases worldwide. AD brain is featuring of extracellular amyloid-β (Aβ) plaques and intraneuronal neurofibrillary (Tau) tangles (8). Both of abnormal structures are highly insoluble and stacked amyloid fibrils. Aβ plaque is derived from proteolytic processing of a transmembrane protein, the amyloid precursor protein (APP). APP is cleaved by β- and γ-secretases to produce Aβ peptide. Evidence has shown that excessive Aβ production induces neurotoxicity, triggering neuronal tangle formation, and neuron loss in the plaques deposited brain regions. This process is believed as the pathogenic mechanism in both familial and sporadic AD. The second risk factor in AD is the microtubule-associate protein Tau. Tau stabilizes microtubule network, regulates axonal integrity and axonal transport. Disruption of microtubule might be caused by the loss of function of Tau through aberrantly hyperphosphorylation. Hyperphosphorylated Tau isoforms as the main component of neurofibrillary tangles (NFTs) and neuropil threads observed in AD brain. At least 19 amino acids are phosphorylated and correlate with AD severity. Accordingly, high-abundance phosphor-Tau (pTau-217) isoform observed in cerebrospinal fluid (CSF) and plasma was used as an early diagnostic marker of AD.

Parkinson disease (PD) is a progressive neurodegenerative disorder primarily affected motor system, usually occurred of rigidity, hypokinesia and tremor. Non-motor symptoms also developed as the disease worsens, including cognitive changes. PD is caused predominantly by the loss of dopaminergic neurons in the substantia nigra (SN), a basal ganglia structure located in the midbrain. The signature pathology of the PD is lewy bodies (LBs) (9). Misfolded alpha-synuclein (α-Syn) as the major component of the LBs observed in sporadic PD; and that mutations in the α-Syn are associated with some rare familiar PD. The second risk factor for PD is the Leucine-rich repeat kinase 2 (LARRK2). LARRK2 gene mutation accounts for 5% of familiar PD as well as 3% of sporadic cases. LRRK2 harbored G2019 mutation is more susceptible to forming α-Syn inclusions that links LRRK2 with protein-misfolding pathology. Currently, the diagnostic process often takes over a year to complete, and currently no approved methods is available for early diagnostic and monitoring disease progression.

b-isox, biotinylated isoxazole (6-(5-(Thiophen-2-yl) isoxazole-3-carboxamido) hexyl 5-((3aS,4S,6aR)-2-oxohexahydro-1H-thieno[3,4-d]imidazol-4-yl) pentanoate), is a small molecule known to precipitate RNA-binding proteins enriched in stress granules and RNA granules. Most of the proteins precipitated by b-isox contained low-complexity (LC) domain, which is responsible for the interaction with b-isox. The LC domains are intrinsic disordered structure and self-interactions. This novel type of self-interaction domain transiently forms a cross-β polymeric condensed phase to perform crucial biological processes, including DNA transcription and replication, chromatin remodeling, nuclear pore passage, signal transduction, synaptic transmission, and cytoskeleton regulation by homotypic-or heterotypic cross-β multimeric interactions. In contrast to pathological cross-β aggregate stacked in-register, the physiological cross-β multimers are loose and reversible (10, 11, 12, 13).

Currently, the cost-effective biomarker testing and noninvasive diagnostic techniques for detection of pathophysiology in clinical and presymptomatic stage of conformational diseases and proteinopathies, particularly neurodegenerative disease, are unmet medical need. The present invention addresses this need and other needs.

### BRIEF SUMMARY OF THE INVENTION

Methods for advances made in the differential diagnostics, real-time monitoring pathophysiology, pharmacoresponse analysis, presymptomatic diagnostics, and subtyping of neurodegenerative diseases by detecting the presence of b-isox precipitates or the level of b-isox-captured proteins from plasma and CSF of patients. Example of neurodegenerative diseases includes, but not limited to, ALS, AD, parkinson disease dementia (PDD), dementia with Lewy body (DLB), parkinson disease without dementia (PDnD), multiple system atrophy (MSA), spinal muscular atrophy (SMA), limbic predominant age related TDP-43 encephalopathy neuropathological change (LATE-NC) and frontotemporal dementia (FTLD).

Example of b-isox precipitated proteins for ALS diagnosis includes, but not limited to, p-TDP-43, SOD1, GR repeat protein (poly (GR)), GA repeat protein (poly(GA)), GP repeat protein (poly(GP)), carbonic anhydrase 1 (CA1), cluster of differentiation 14 (CD14), myosin light chain 12B (MYL12B), peroxiredoxin 2 (PRDX2), stomatin (STOM), profilin1 (PFN1), β-actin (ACTB), glucose transporter 1 (GLUT-1), survival of motor neuron 1 (SMN), and annexin A5 (ANXA5).

Example of b-isox precipitated proteins for PD diagnosis includes, but not limited to, p-TDP-43, CA1, CD14, PRDX2, STOM, ANXA5, synuclein, cellular adhesion molecule L1 like (CHL1), RUVB like AAA ATPase1 (RUVBL1), neural EGFL like 2 (NELL2), ankyrin-1 (ANK1), and neuronal cell adhesion molecule (NrCAM).

Example of b-isox precipitated proteins for AD diagnosis includes, but not limited to, amyloid beta, phospho-TDP-43, TDP-43, Tau, STOM, and ANK1.

Methods for advances made in presymptomatic diagnostic of SOD1 inherited and sporadic ALS by detecting the level of b-isox-captured GR repeat proteins from plasma and CSF of patients.

A method for detecting a human disease in a subject, comprises detecting a presence of biofluid isoxazole-precipitates in a biofluid sample of the subject. In some embodiments, the method of claim 1, wherein the isoxazole in the isoxazole-precipitates is biotin-isoxazole, (6-(5-(Thiophen-2-yl)isoxazole-3-carboxamido)hexyl 5-((3aS,4S,6aR)-2-oxohexahydro-1H-thieno[3,4-d]imidazol-4-yl)pentanoate) or its salt or an analog thereof.

In some embodiments, the human disease is Amyotrophic lateral sclerosis (ALS), Alzheimer's disease (AD), parkinson disease dementia (PDD), parkinson disease no dementia (PDnD), dementia with Lewy body (DLB), multiple system atrophy (MSA), spinal muscular atrophy (SMA), and limbic predominant age related TAR DNA-binding protein 43 (TDP-43) encephalopathy neuropathological change (LATE-NC) or frontotemporal dementia (FTLD).

In some embodiments, the biofluid is cerebrospinal fluid (CSF) or plasma.

A method for detecting a human conformational disease in a subject, comprises detecting a presence of biofluid isoxazole-captured proteins in a biofluid sample of the subject.

In some embodiments, the isoxazole is biotin-isoxazole (6-(5-(Thiophen-2-yl)isoxazole-3-carboxamido)hexyl 5-((3aS,4S,6aR)-2-oxohexahydro-1H-thieno[3,4-d]imidazol-4-yl)pentanoate) or its analogous.

In some embodiments, the human conformational disease is Amyotrophic lateral sclerosis (ALS), Alzheimer's disease (AD), parkinson disease dementia (PDD), parkinson disease no dementia (PDnD), dementia with Lewy body (DLB), multiple system atrophy (MSA), spinal muscular atrophy (SMA), and limbic predominant age related TAR DNA-binding protein 43 (TDP-43) encephalopathy neuropathological change (LATE-NC) or frontotemporal dementia (FTLD), or diabetes.

In some embodiments, the isoxazole-captured proteins are detected by a polypeptide.

In some embodiments, the polypeptide is (a) an antibody, or (b) an immunoglobulin chain, or a binding domain thereof which binds to the isoxazole-captured proteins. In some embodiments, the human conformational diseases is ALS, and wherein the polypeptide for detecting ALS is an antibody against SOD1, C9orf72 dipeptide repeats, PFN1, PRDX2, phospho-TDP-43, CA1, MYL12B, CD14, ANXA5, STOM, SMN, ACTB, or GLUT1. In some embodiments, the human conformational diseases is AD, and wherein polypeptide for detecting AD is an antibody against APP, phospho-TDP-43, TDP-43, Tau, STOM, or ANK1. In some embodiments, the human conformational diseases is PD, and wherein polypeptide for detecting PD is an antibody against synuclein, CHL1, NELL2, p-TDP-43, NrCAM, ANK1, STOM, PRDX2, CA1, CD14, and RUVBL1. In some embodiments, the biofluid is plasma and CSF.

A method for detecting conformational disease in a subject, comprises the step of detecting the biofluid level of proteins with cross-β structure in the sample of the subject.

In some embodiments, the biofluid is plasma and CSF. In some embodiments, the proteins with cross-β structure is detected by a combination of a cross-β recognized probe and a polypeptide. In some embodiments, the conformational disease is ALS, AD, PDD, PDnD, DLB, MSA, SMA, FTLD, diabetes, schizophrenia, cancer, aging and limbic predominant age related TDP-43 encephalopathy neuropathological change (LATE-NC) or diabetes.

The use of a reagent for detecting a b-isox captured complex in the manufacture of a kit to evaluate the risks of developing conformational disease or the progression of conformational diseases.

A method for detecting aging and conformational disease in a subject, comprises the step of detecting the presence of low complexity protein' complex in the sample of the subject.

A method for detecting sporadic ALS in a subject, comprises the step of detecting the presence of dipeptide repeat proteins in the sample of the subject.

In some embodiments, the dipeptide repeat protein is poly (GR), poly (GP), and poly (GA).

A method for detecting sporadic ALS and SOD1 inherited ALS at the presymptomatic and prodromal stage in a subject, comprises the step of detecting the presence of dipeptide repeat proteins in the sample of the subject.

In some embodiments, the dipeptide repeat protein is poly (GR).

The terms “invention,” “the invention,” “this invention” and “the present invention” used in this patent are intended to refer broadly to all of the subject matter of this patent and the patent claims below. Statements containing these terms should be understood not to limit the subject matter described herein or to limit the meaning or scope of the patent claims below. Embodiments of the invention covered by this patent are defined by the claims below, not this summary. This summary is a high-level overview of various aspects of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification, any or all drawings and each claim.

The invention will become more apparent when read with the accompanying figures and detailed description which follow.

It is to be understood that both the foregoing general description and the following detailed description are exemplary and explanatory only and are not restrictive of the invention, as claimed.

The accompanying drawings, which are incorporated in and constitute a part of this specification, illustrate one (several) embodiment(s) of the invention and together with the description, serve to explain the principles of the invention.

### BRIEF DESCRIPTION OF THE DRAWINGS

Illustrative embodiments of the present invention are described in detail below with reference to the following Figures:

FIG. 1 is an assembly of images illustrating an analysis of b-isox precipitates from the plasma of healthy controls and patients with ALS. Panel (a) contains an assembly of images of chemical precipitates isolated from the plasma of healthy individuals and ALS patients. The arrow indicates b-isox-precipitated pellets. The bottom of each picture is the ruler used to measure the size of the chemical precipitate (mm). Panel (b) contains a statistical analysis of time course of chemical precipitates isolated from the plasma of healthy individuals and ALS patients. Arrowhead indicates optimal condition. Panel (c) contains a statistical analysis of plasma volume of chemical precipitates isolated from the plasma of healthy individuals and ALS patients. Arrowhead indicates optimal condition. Panel (d) contains a statistical analysis of the size of b-isox precipitates isolated from 100 μl plasma of normal, ALS, AD, and PD patients. All the data are presented as the mean with SD (n of Normal, ALS, AD, PD=31, 65, 11, 14, respectively.). ****P<0.0001 by Mann-Whitney test. Panel (e) contains an analysis of longitudinal tracking of b-isox precipitates from two patients with ALS. The size of b-isox precipitates from the plasma of two ALS patients was indicated with months.

FIG. 2 is an assembly of images illustrating b-isox ELISA and its applications in ALS. Panel (a) contains a protocol flowchart of b-isox ELISA. Panel (b) contains an assembly of images illustrating the levels of SOD1 in b-isox precipitates isolated from the plasma of normal controls, SMA mice and ALS mice by b-isox ELISA. *P<0.05 by the Mann-Whitney test. Panel (c) contains an assembly of images illustrating the plasma levels of b-isox captured SOD1 in a patient with a SOD1 G93A mutation. Panel (d) contains an assembly of images illustrating SOD1 subtyping. b-isox ELISA shows the levels of SOD1 in the plasma of normal controls and patients with ALS. All the data are presented as the mean and SD (n of Normal and ALS=38 and 161, respectively.). ****P<0.0001 by the Mann-Whitney test. Panel (e) contains an assembly of images illustrating TDP-43 subtyping. b-isox ELISA shows the levels of p-TDP-43 in the plasma of normal controls and patients with ALS. All the data are presented as the mean and SD (n of Normal and ALS=17 and 115, respectively.). ****P<0.0001 by the Mann-Whitney test. Panel (f) contains an assembly of images illustrating the biomarkers' profiling of a patient with TDP-43 proteinopathy by b-isox ELISA. Panel (g) contains an assembly of images illustrating longitudinal tracking of the associations between p-TDP-43 levels, b-isox-precipitates, and ALSFRS-R in an ALS patient with TDP-43 proteinopathies. Panel (h) contains an assembly of images illustrating the analysis of plasma level of GR repeat proteins in ALS. b-isox ELISA shows the levels of GR repeat proteins in the plasma of normal controls and patients with ALS. All the data are presented as the mean and SD (n of Normal and ALS=17 and 111, respectively.). ****P<0.0001 by the Mann-Whitney test. Panel (i) contains an assembly of images illustrating the biomarkers' profiling of a patient with the poly (GR) proteinopathies by b-isox ELISA. Panel (j) contains an assembly of images illustrating longitudinal tracking of the association between GR repeat proteins levels, b-isox precipitates, and ALSFRS-R in an sporadic ALS patient with poly (GR) proteinopathies.

FIG. 3 is an assembly of images illustrating identification and validation of newly discovered biomarkers for ALS by b-isox ELISA. Panel (a) contains an assembly of images illustrating a schematic diagram of the experimental design for the discovery of novel plasma biomarkers for neurodegenerative diseases. Panel (b) contains an assembly of images illustrating the plasma levels of CA1 in healthy individuals (H) and patients with ALS. All the data are presented as the mean and SD (n of H and ALS=20 and 106, respectively.). *P<0.05 by the Mann-Whitney test. Panel (c) contains an assembly of images illustrating b-isox ELISA shows the levels of CD14 in the plasma of healthy controls (H) and patients with ALS. All the data are presented as the mean and SD (n of H and ALS=25 and 95, respectively.). *P<0.05 by the Mann-Whitney test. Panel (d) contains an assembly of images illustrating b-isox ELISA shows the plasma levels of MYL12B in healthy controls (H) and patients with ALS. All the data are presented as the mean and SD (n of H and ALS=18 and 91, respectively.). *P<0.05 by the Mann-Whitney test. Panel (e) contains an assembly of images illustrating b-isox ELISA shows the plasma levels of PRDX2 in healthy controls (H) and patients with ALS. All the data are presented as the mean and SD (n of H and ALS=22 and 103, respectively.). *P<0.05 by the Mann-Whitney test. Panel (f) contains an assembly of images illustrating b-isox ELISA shows the plasma levels of STOM in healthy controls (H) and patients with ALS. All the data are presented as the mean and SD (n of H and ALS=17 and 97, respectively.). *P<0.05 by the Mann-Whitney test. Panel (g) contains an assembly of images illustrating the plasma levels of GLUT1 in healthy controls (H) and patients with ALS. All the data are presented as the mean and SD (n of H and ALS=12 and 53, respectively.). *P<0.05 by the Mann-Whitney test. Panel (h) contains an assembly of images illustrating the plasma levels of SMN in healthy controls (H) and patients with ALS. All the data are presented as the mean and SD (n of H and ALS=14 and 58, respectively.). *P<0.05 by the Mann-Whitney test. Panel (i) contains an assembly of images illustrating the plasma levels of beta-Actin in healthy controls (H) and patients with ALS. All the data are presented as the mean and SD (n of H and ALS=8 and 13, respectively.). *P<0.05 by the Mann-Whitney test. Panel (j) contains an assembly of images illustrating the plasma levels of PFN1 in healthy controls (H) and patients with ALS. All the data are presented as the mean and SD (n of H and ALS=15 and 43, respectively.). Panel (k) contains an assembly of images illustrating the plasma levels of poly (GP) in healthy controls (H) and patients with ALS. All the data are presented as the mean and SD (n of H and ALS=8 and 13, respectively.). *P<0.05 by the Mann-Whitney test. Panel (l) contains an assembly of images illustrating the plasma levels of poly (GA) in healthy controls (H) and patients with ALS. All the data are presented as the mean and SD (n of H and ALS=15 and 43, respectively.). *P<0.05 by the Mann-Whitney test.

FIG. 4 is an assembly of images illustrating an analysis of the plasma levels of ALS biomarkers during disease progression stage. Panel (a) contains an assembly of images illustrating the plasma levels of eleven biomarkers, MYL12B, STOM, CA1, p-TDP-43, GR repeat proteins, SOD1, CD14, PRDX2, GLUT1, ANXA5, and SMN at individual ALS stages were measured. The percentages on top represent the patients with levels higher than the cutoff values indicated by dot line. ALSFRS-R: stage I (score 40-48), II (score 30-39), III (score 20-29), IV (score 10-19). Panels (b) contains the assembly of images illustrating a trend chart of the three biomarkers, including MYL12B, CD14 and PRDX2 in the progression of ALS. Panels (c) contains the assembly of images illustrating the changes of CA1, pTDP-43, SOD1 and GR repeat proteins in the progression of ALS. Panels (d) contains the assembly of images illustrating the changes of ANXA5 in the progression of ALS.

FIG. 5 is an assembly of images illustrating longitudinal tracking of ALS biomarkers. Panel (a-c) contains an assembly of images illustrating three examples of longitudinal tracking of eight biomarkers, MYL12B, CD14, STOM, PRDX2, p-TDP-43, CA1, SOD1, and GR repeat proteins, in patients with ALS.

FIG. 6 is an assembly of images illustrating a pharmacoresponse analysis of edaravone in ALS patients by b-isox ELISA. Two examples of pharmacometric measurements of edaravone treatment. Arrows indicated the treatments with edaravone.

FIG. 7 is an assembly of images illustrating GR repeat proteins as presymptomatic biomarker for SOD1 inherited and sporadic ALS. Panel (a) contains an assembly of images illustrating profiles of a combination of measurements of plasma biomarkers MYL12B, STOM, CA1, p-TDP-43, GR repeat proteins, SOD1, CD14, and PRDX2 for patients with ALSFRS-R scores above 42 by b-isox-based ELISA. Panel (b) contains an assembly of images illustrating a b-isox ELISA showing the plasma levels of GR repeat proteins in ALS, AD and PD. All the data are presented as the mean and SD. Panel (c) contains an assembly of images illustrating a b-isox ELISA showing the plasma levels of SOD1, p-TDP-43, CD14, GR repeat proteins, and poly (GA) in normal controls and individual with C9orf72 inherited ALS. Panel (d) contains an assembly of images illustrating the levels of GR repeat proteins in the plasma of normal controls and ALS mice in the indicated months by b-isox-ELISA. All the data are presented as the mean and SD. *P<0.05 by Mann-Whitney test.

FIG. 8 is an assembly of images illustrating a validation of newly identified PD biomarkers by b-isox ELISA. Panel (a) contains an assembly of images illustrating plasma levels of synuclein in healthy individuals (H) and patients with PD. All the data are presented as the mean and SD (n of H and PD=16 and 51, respectively.). ****P<0.0001 by Mann-Whitney test. Panel (b) contains an assembly of images illustrating ELISA shows the levels of ANXA5 in the plasma of healthy controls (H) and patients with PD. All the data are presented as the mean and SD (n of H and PD=16 and 30, respectively.). **P<0.01 by the Mann-Whitney test. Panel (c) contains an assembly of images illustrating ELISA shows the levels of CA1 in the plasma of healthy controls (H) and patients with PD. All the data are presented as the mean and SD (n of H and PD=20 and 20, respectively.). ****P<0.0001 by Mann-Whitney test. Panel (d) contains an assembly of images illustrating ELISA shows plasma levels of CD14 in healthy controls (H) and patients with PD. All the data are presented as the mean and SD (n of H and PD=28 and 20, respectively.). ****P<0.0001 by Mann-Whitney test. Panel (e) contains an assembly of images illustrating ELISA shows plasma levels of CHL1 in healthy controls (H) and patients with PD. All the data are presented as the mean and SD (n of H and PD=12 and 42, respectively.). ***P<0.001 by the Mann-Whitney test. Panel (f) contains an assembly of images illustrating ELISA shows plasma levels of NrCAM in healthy controls (H) and patients with PD. All the data are presented as the mean and SD (n of H and PD=4 and 34, respectively.). ****P<0.0001 by Mann-Whitney test. Panel (g) contains an assembly of images illustrating the plasma levels of PRDX2 in healthy controls (H) and patients with PD. All the data are presented as the mean and SD (n of H and PD=22 and 13, respectively.). ****P<0.0001 by Mann-Whitney test. Panel (h) contains an assembly of images illustrating plasma levels of p-TDP-43 in healthy controls (H) and patients with PD. All the data are presented as the mean and SD (n of H and PD=17 and 46, respectively.). ****P<0.0001 by Mann-Whitney test. Panel (i) contains an assembly of images illustrating plasma levels of RUVBL1 in healthy controls (H) and patients with PD. All the data are presented as the mean and SD (n of H and PD=7 and 13, respectively.). **P<0.01 by the Mann-Whitney test. Panel (j) contains an assembly of images illustrating plasma levels of STOM in healthy controls (H) and patients with PD. All the data are presented as the mean and SD (n of H and PD=17 and 33, respectively.). ****P<0.0001 by Mann-Whitney test.

FIG. 9 is an assembly of images illustrating the biomarkers' profiling of patients with PDnD (a), PDD (b), MSA (c) and DLB (d) by b-isox-ELISA assay.

FIG. 10 is an assembly of images illustrating a validation of known and newly identified AD biomarkers by b-isox-ELISA. Panel (a) contains an assembly of images illustrating plasma levels of amyloid beta, tau, TDP-43, p-TDP-43, ANK1 and STOM in healthy individuals (H) and patients with AD. All the data are presented as the mean and SD (n of H and AD=12, 4, 2, 17, 14, 2 and 13, 6, 10, 12, 13, 9 respectively.). *P<0.05 by the Mann-Whitney test. Panel (b) contains an assembly of images illustrating sensitivity comparison of amyloid β, ANK1, and STOM by b-isox-ELISA.

### DESCRIPTION OF EMBODIMENTS

Reference will now be made in detail to the present embodiment(s) (exemplary embodiments) of the invention, an example(s) of which is (are) illustrated in the accompanying drawings. Wherever possible, the same reference numbers will be used throughout the drawings to refer to the same or like parts.

Methods for the detection of conformational diseases and proteinopathies, such as neurodegenerative diseases, are described herein by using a small-molecule to generate visual precipitates.

In addition, applicants identified novel and specific biofluid biomarkers for differential diagnosis and monitoring pathophysiology in patients with neurodegenerative diseases, including ALS, AD and PD.

Also described are novel methods for the plasma diagnosis of neurodegenerative diseases. The novel method named b-isox-ELISA, is combination of b-isox chemical-precipitation and immunoassay with specific biomarkers of ALS, AD or PD. b-isox ELISA can be used to screen the risks of ALS, AD and PD. This invention can be used to distinguish between AD, TDP-43 proteinopathies, PD, and dementia with Lewy bodies, ALS subtyping, real-time readout of pharmacoresponse, and monitoring the relief of pathological burden of misfolded disease proteins in clinical trial, preclinical diagnosis and clinical practice.

### Definitions

As employed above and throughout the disclosure, the following terms, unless otherwise indicated, shall be understood to have the following meanings.

As used herein, the singular forms “a,” “an,” and “the” include the plural reference unless the context clearly indicates otherwise.

### Representative Embodiments

Embodiment 1. A method for detecting a human disease in a human subject, comprises, optionally obtaining a biofluid sample from the subject, adding an isoxazole to the obtained biofluid sample to form a biofluid isoxazole composition in the biofluid sample, and detecting a presence of the biofluid isoxazole composition.

Embodiment 2. The method of Embodiment 1, wherein the isoxazole is biotin-isoxazole, (6-(5-(Thiophen-2-yl)isoxazole-3-carboxamido)hexyl 5-((3aS,4S,6aR)-2-oxohexahydro-1H-thieno[3,4-d]imidazol-4-yl)pentanoate), or its salt or an analog thereof, especially biotin-isoxazole or its salt and very especially biotin-isoxazole.

Embodiment 3. The method of Embodiments 1-2, wherein the biofluid sample is urine, whole blood, plasma, or serum, cerebrospinal fluid (CSF), saliva, or mucosa, such as, urine, saliva, CSF or plasma, and especially CSF or plasma. In some embodiments, the biofluid sample is in the form of a biological fluid such as urine, whole blood, plasma, or serum, cerebrospinal fluid (CSF), saliva, or mucosa, and optionally, the biofluid sample is further processed, e.g., to remove some components, e.g., by techniques to enrich components such as proteins by chemical precipitation. In some embodiments, the biofluid sample is blood, plasma, or serum, CSF, urine or saliva. In some embodiments, the biofluid sample is plasma or CSF.

Embodiment 4. The method of Embodiments 1-3, wherein the human disease is Amyotrophic lateral sclerosis (ALS), Alzheimer's disease (AD), parkinson disease dementia (PDD), parkinson disease no dementia (PDnD), dementia with Lewy body (DLB), multiple system atrophy (MSA), spinal muscular atrophy (SMA), and limbic predominant age related TAR DNA-binding protein 43 (TDP-43) encephalopathy neuropathological change (LATE-NC), stroke, cerebral amyloid angiopathy (CAA), frontotemporal dementia (FTLD), diabetes, cancer, infectious disease, huntington disease, schizophrenia, aging-associated disease, or protienopathies, especially wherein the human disease is Amyotrophic lateral sclerosis (ALS), Alzheimer's disease (AD), parkinson disease dementia (PDD), parkinson disease no dementia (PDnD), dementia with Lewy body (DLB), multiple system atrophy (MSA), spinal muscular atrophy (SMA), and limbic predominant age related TAR DNA-binding protein 43 (TDP-43) encephalopathy neuropathological change (LATE-NC), stroke, cerebral amyloid angiopathy (CAA), or frontotemporal dementia (FTLD).

Embodiment 5. The method of Embodiments 1-4, further comprises thereafter treating the human disease or thereafter changing an existing treatment based on the detecting the presence of the biofluid isoxazole composition, such as a treatment including pharmaceutical therapy for the human disease. In some embodiments, the method further comprises diagnosing the human disease.

Embodiment 6. The method of Embodiments 1-5, wherein the concentration of isoxazole ranges from 0.075 mM to 0.225 mM, preferably from 0.100 mM to 0.200 mM, in the biofluid sample.

Embodiment 7. The method of Embodiments 1-6, wherein the biofluid isoxazole composition is in a precipitate.

Embodiments 8. The method of Embodiment 7, wherein the human disease is Amyotrophic lateral sclerosis (ALS), Alzheimer's disease (AD), parkinson disease dementia (PDD), parkinson disease no dementia (PDnD), dementia with Lewy body (DLB), multiple system atrophy (MSA), spinal muscular atrophy (SMA), and limbic predominant age related TAR DNA-binding protein 43 (TDP-43) encephalopathy neuropathological change (LATE-NC), stroke, cerebral amyloid angiopathy (CAA), or frontotemporal dementia (FTLD).

Embodiment 9. The method of Embodiment 8, wherein the human disease is ALS, AD, PDD, DLB, MSA or PDnD.

Embodiment 10. The method of Embodiment 9, wherein the human disease is ALS, such as sporadic ALS.

Embodiment 11. The method of Embodiments 7-10, further comprising monitoring the size of the precipitate to monitor the progression of ALS in the subject.

Embodiment 12. The method of Embodiments 1-6, further comprises adding a polypeptide to biofluid isoxazole composition in the biofluid sample to facilitate detection by an immune assay, such as a blot assay, a chemiluminescence immunoassay, an enzyme-linked immunosorbent assay (ELISA), a light scattering immunoassay, a radiolabeled immunoassay, in particular, ELISA or a Western blot.

Embodiment 13. The method of Embodiment 12, wherein the human conformational disease is Amyotrophic lateral sclerosis (ALS), Alzheimer's disease (AD), parkinson disease dementia (PDD), parkinson disease no dementia (PDnD), dementia with Lewy body (DLB), multiple system atrophy (MSA), spinal muscular atrophy (SMA), and limbic predominant age related TAR DNA-binding protein 43 (TDP-43) encephalopathy neuropathological change (LATE-NC), stroke, cerebral amyloid angiopathy (CAA), or frontotemporal dementia (FTLD).

Embodiment 14. The method of Embodiments 12-13, wherein the polypeptide is (a) an antibody, or (b) an immunoglobulin chain, or a binding domain thereof which binds to the biofluid isoxazole composition.

Embodiments 15. The method of Embodiment 14, wherein the human diseases is ALS, and wherein the polypeptide for detecting ALS is an antibody against SOD1, C9orf72 dipeptide repeats, PFN1, PRDX2, phospho-TDP-43, CA1, MYL12B, CD14, ANXA5, STOM, SMN, ACTB, or GLUT1, such as SOD1, MYL12B, CD14 and p-TDP-43, and especially SOD1 and p-TDP-43.

Embodiment 16. The method of Embodiment 14, wherein the human diseases is AD, and wherein polypeptide for detecting AD is an antibody against APP, phospho-TDP-43, TDP-43, Tau, STOM, or ANK1.

Embodiment 17. The method of Embodiment 14, wherein the human diseases is PD, and wherein polypeptide for detecting PD is an antibody against synuclein, CHL1, NELL2, p-TDP-43, NrCAM, ANK1, STOM, PRDX2, CA1, CD14, and RUVBL1.

Embodiment 18. The method of Embodiments 15-17, wherein biofluid is plasma or CSF.

Embodiment 19. The method of Embodiments 12-18, wherein the method is ELISA, such as direct, indirect, sandwich, or competitive ELISA.

Embodiment 20. Use of an isoxazole to detect a human disease in any method of Embodiments 1-19.

Embodiment 21. A method for detecting a human disease in a human subject, comprises, detecting a presence of a biomarker from an obtained biofluid sample, and optionally obtaining a biofluid sample from the subject to obtain the obtained biofluid sample, and wherein the human disease is selected from sporadic Amyotrophic lateral sclerosis (ALS), Alzheimer's disease (AD), parkinson disease dementia (PDD), and parkinson disease no dementia (PDnD); wherein, for sporadic ALS, the biomarker is selected from C9orf72 dipeptide repeats, PRDX2, CA1, MYL12B, CD14, ANXA5, STOM, SMN, and GLUT1; wherein, for AD, the biomarker is selected from STOM and ANK1, and wherein for PDD or PDnD, the biomarker is selected from CHL1, NELL2, NrCAM, ANK1, STOM, PRDX2, CA1, CD14, and RUVBL1.

Embodiment 22. The method of Embodiment 21, wherein the obtained biofluid sample is urine, whole blood, plasma, or serum, cerebrospinal fluid (CSF), saliva, or mucosa, such as, urine, saliva, CSF or plasma, and especially CSF or plasma. The biofluid sample includes those in Embodiment 3.

Embodiment 23. The method of Embodiments s 21-22, wherein the human disease is sporadic ALS.

Embodiment 24. The method of Embodiments 21-22, wherein the human disease is AD.

Embodiment 25. The method of Embodiments 21-22, wherein the human disease is DLB, MSA, PDD or PDnD.

Embodiment 26. The method of Embodiments 21-25, further comprising thereafter treating the human subject for the human disease or thereafter changing an existing treatment of the human subject for the human disease based on the detecting the presents of the biofluid isoxazole composition, such as a treatment including pharmaceutical therapy for the human disease, and optionally further comprising diagnosing the human disease in the human subject.

Embodiment 27. The method of Embodiments 21-26, further comprising adding a polypeptide to the obtained biofluid sample to facilitate detection by an immune assay, such as a blot assay, a chemiluminescence immunoassay, an enzyme-linked immunosorbent assay (ELISA), a light scattering immunoassay, a radiolabeled immunoassay, in particular, ELISA or a Western blot.

Embodiment 28. The method of Embodiment 27, wherein the polypeptide is (a) an antibody, or (b) an immunoglobulin chain, or a binding domain thereof which binds to the biomarker.

Embodiment 29. The method of Embodiment 28, wherein the human diseases is sporadic ALS, and wherein the polypeptide for detecting sporadic ALS is an antibody against C9orf72 dipeptide repeats, PRDX2, CA1, MYL12B, CD14, ANXA5, STOM, SMN, or GLUT1, such as C9orf72 dipeptide repeats.

Embodiment 30. The method of Embodiment 28, wherein the human diseases is AD, and wherein polypeptide for detecting AD is an antibody against STOM or ANK1.

Embodiment 31. The method of Embodiment 28, wherein the human diseases is DLB, MSA, PDD or PDnD, and wherein polypeptide for detecting PD is an antibody against CHL1, NrCAM, ANK1, STOM, PRDX2, CA1, CD14, or RUVBL1.

Embodiment 32. The method of Embodiments 29-31, wherein biofluid is plasma or CSF.

Embodiment 33. The method of Embodiment 27-32, wherein the method is ELISA, such as direct, indirect, sandwich, or competitive ELISA.

Embodiment 34. Use of the biomarker to detect the human disease in any method of Embodiments 21-33.

Embodiment 35. A method for detecting sporadic Amyotrophic lateral sclerosis (ALS) in a human subject, comprises, detecting a presence of a dipeptide repeat protein from an obtained biofluid sample, and optionally obtaining a biofluid sample from the subject to obtain the obtained biofluid sample.

Embodiment 36. The method of Embodiment 35, wherein the biofluid sample is urine, whole blood, plasma, or serum, cerebrospinal fluid (CSF), saliva, or mucosa, such as, urine, saliva, CSF or plasma, and especially CSF or plasma.

Embodiment 37. The method of Embodiments 35-36, wherein the dipeptide repeat protein is selected from poly (GR), poly (GP), and poly (GA).

Embodiment 38. The method of Embodiments 35-37, further comprises thereafter treating the human subject for the human disease or thereafter changing an existing treatment of the human subject for the human disease based on the detecting the presents of the biofluid isoxazole composition, such as a treatment including pharmaceutical therapy for the human disease, and optionally further comprising diagnosing the human disease in the human subject.

Embodiment 39. The method of Embodiments 35-38, further comprising adding a polypeptide to the obtained biofluid sample to facilitate detection by an immune assay, such as a blot assay, a chemiluminescence immunoassay, an enzyme-linked immunosorbent assay (ELISA), a light scattering immunoassay, a radiolabeled immunoassay, in particular, ELISA or a Western blot.

Embodiment 40. The method of Embodiment 39, wherein the polypeptide is (a) an antibody, or (b) an immunoglobulin chain, or a binding domain thereof which binds to the biomarker.

Embodiment 41. The method of Embodiment 40, wherein the human diseases is sporadic ALS, and wherein the polypeptide for detecting sporadic ALS is an antibody against dipeptide repeat proteins, PRDX2, CA1, MYL12B, CD14, ANXA5, STOM, SMN, or GLUT1, such as C9orf72 dipeptide repeats.

Embodiment 42. The method of Embodiment 39-41, wherein biofluid is plasma or CSF.

Embodiment 43. The method of Embodiments 39-42, wherein the method is ELISA, such as direct, indirect, sandwich, or competitive ELISA.

Embodiment 44. Use of the dipeptide repeat protein to detect sporadic ALS in any method of Embodiments 35-43.

Embodiment 45. A method for detecting sporadic or SOD1 inherited Amyotrophic lateral sclerosis (ALS) in a human subject at the presymptomatic and prodromal stage, comprises, detecting a presence of a dipeptide repeat protein from an obtained biofluid sample, and optionally obtaining a biofluid sample from the subject to obtain the obtained biofluid sample.

Embodiment 46. The method of Embodiment 45, wherein the biofluid sample is urine, whole blood, plasma, or serum, cerebrospinal fluid (CSF), saliva, or mucosa, such as, urine, saliva, CSF or plasma, and especially CSF or plasma.

Embodiment 47. The method of Embodiments 45-46, wherein the dipeptide repeat protein is poly (GR).

Embodiment 48. The method of Embodiments 45-47, further comprising thereafter treating the human subject for the human disease or thereafter changing an existing treatment of the human subject for the human disease based on the detecting the presents of the biofluid isoxazole composition, such as a treatment including pharmaceutical therapy for the human disease, and optionally further comprising diagnosing the human disease in the human subject.

Embodiments 49. The method of Embodiments 45-48, further comprising adding a polypeptide to the obtained biofluid sample to facilitate detection by an immune assay, such as a blot assay, a chemiluminescence immunoassay, an enzyme-linked immunosorbent assay (ELISA), a light scattering immunoassay, a radiolabeled immunoassay, in particular, ELISA or a Western blot.

Embodiment 50. The method of Embodiment 49, wherein the polypeptide is (a) an antibody, or (b) an immunoglobulin chain, or a binding domain thereof which binds to the biomarker.

Embodiment 51. The method of Embodiment 50, wherein the polypeptide for detecting sporadic or SOD1 inherited ALS is an antibody against dipeptide repeat proteins, PRDX2, CA1, MYL12B, CD14, ANXA5, STOM, SMN, or GLUT1, such as C9orf72 dipeptide repeats.

Embodiment 52. The method of Embodiments 49-51, wherein biofluid is plasma or CSF.

Embodiment 53. The method of Embodiments 49-52, wherein the method is ELISA, such as direct, indirect, sandwich, or competitive ELISA.

Embodiment 54. Use of the dipeptide repeat protein to detect sporadic or SOD1 inherited ALS in any method of Embodiments 45-53.

Embodiment 55. Use of any described substance or composition for diagnosing the human disease in each Embodiment 1-53.

### Methods for Diagnostic, Monitor and Predict Disease Progression of Neurodegenerative Diseases by Detecting b-isox-precipitates from Plasma of Patients

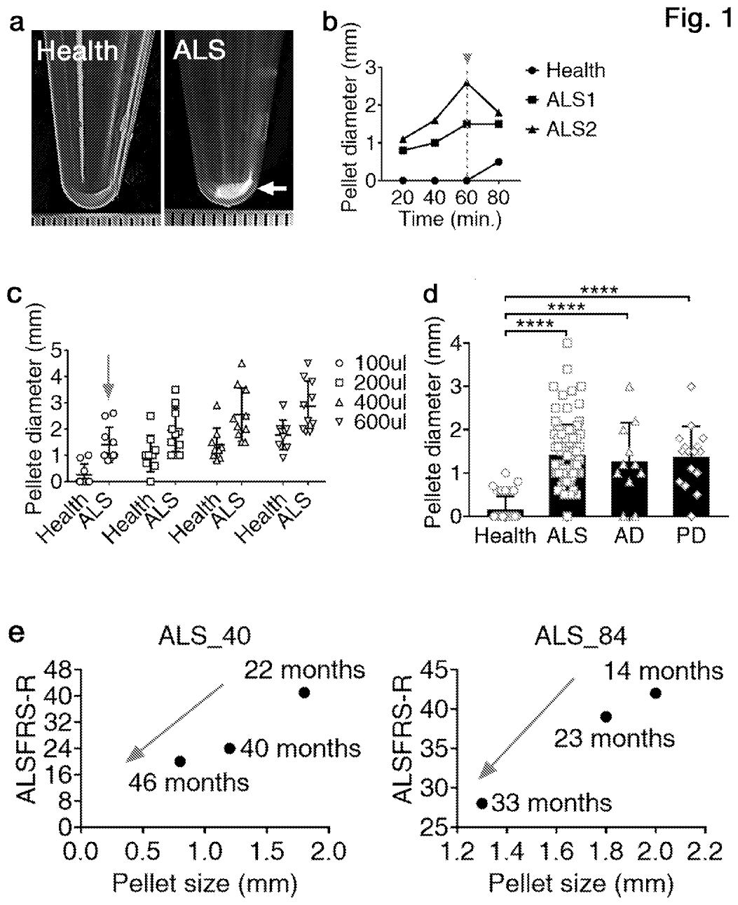

Applicants uses a small-molecule compound, b-isox to generate precipitates, which can be visually observed in samples from patients with ALS, AD and PD, but not in samples from healthy people (FIG. 1). b-isox precipitates accurately discriminated healthy individuals from ALS patients. (FIG. 1A).

Applicants found plasma (0.1 mL) is sufficient for this diagnosis (FIG. 1C), and 60 min. of reaction time reaches the optimal condition (FIG. 1B). The accuracy of our diagnostic for ALS, AD, and PD is 98.4%, 81.8%, 92.8%, respectively (FIG. 1D).

The size of b-isox precipitates is negatively correlated with the functional score (ALSFRS-R), which suggests that b-isox precipitates can be used not only in diagnostics but also for the prediction of future progression to ALS disability (FIG. 1E).

### Methods for Differential- and Presymptomatic- Diagnostic, Subtyping, and Pharmacoresponse Analysis of ALS by Detecting CSF and Plasma b-isox-captured Proteins

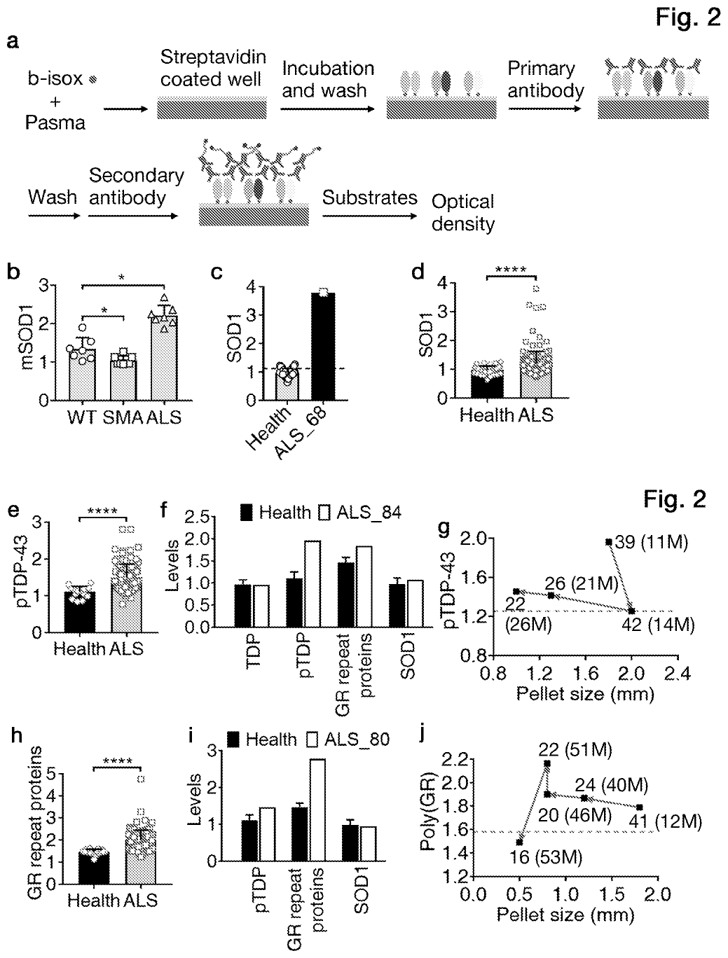

Applicants developed a novel chemical ELISA method, termed b-isox ELISA, for detecting the levels of specific proteins in b-isox precipitates (FIG. 2a).

Applicants found the chemical ELISA detected the levels of specific b-isox captured proteins tested in plasma, and significantly differed between the neurodegenerative disease groups and health controls (FIGS. 2, 3, 9 and 10).

Applicants further found the percentage of patients with high plasma misfolded proteins, including SOD1, p-TDP-43 and C9orf72, corresponds closely to the proportion of patients known from autopsy studies to have their inclusions in the spinal cord, as shown in FIG. 2. These results suggested b-isox-based ELISA can detect the real-time conformational changes of representative known disease-causing proteins, including TDP-43, SOD1, and C9orf72 dipeptide repeat (poly(GR)) proteins, in the plasma of ALS patients.

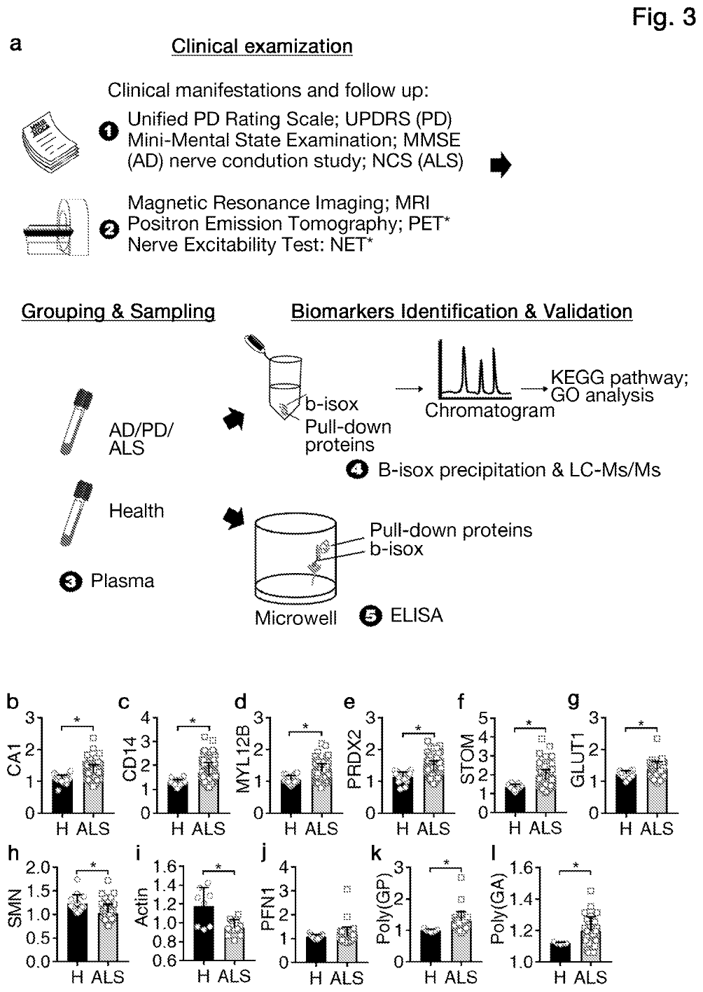

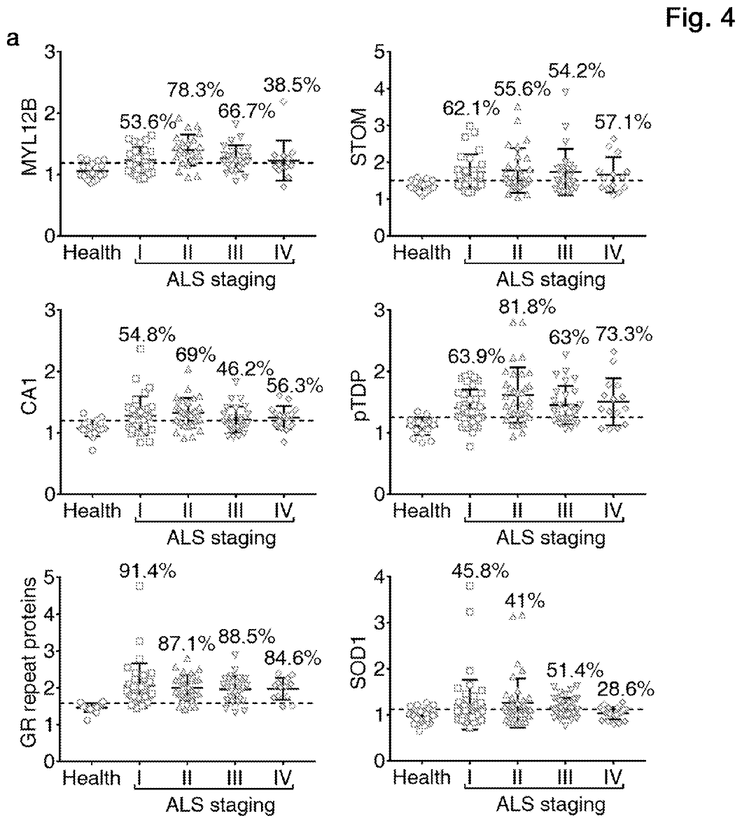

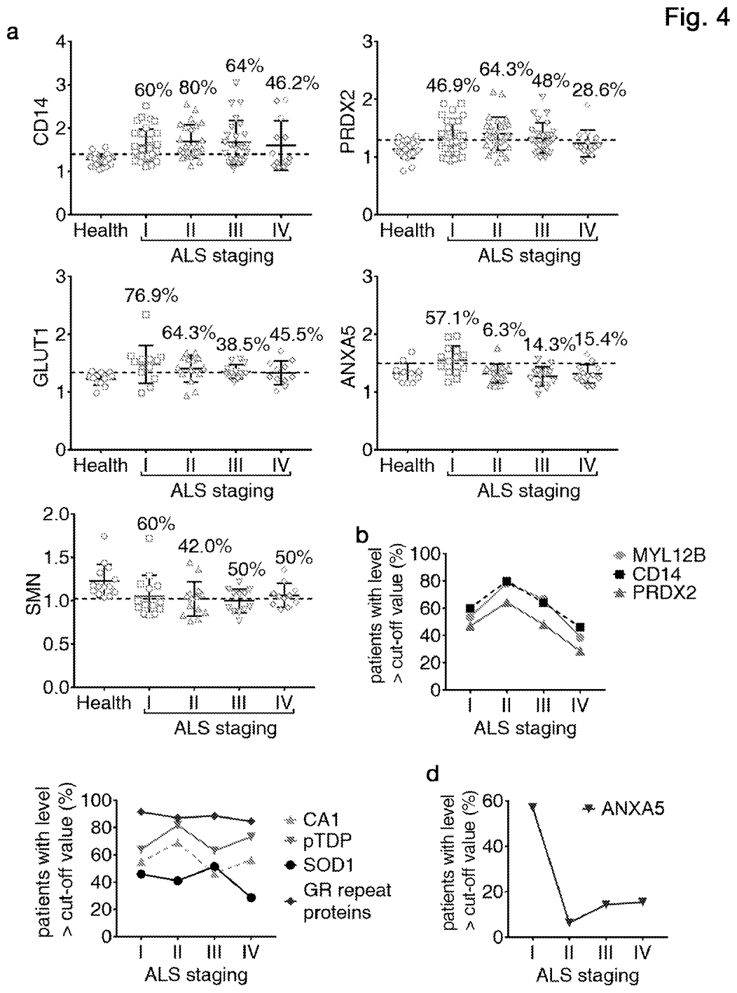

Applicants further identified novel patholophysiology biomarkers of ALS by proteomics analysis of b-isox precipitates, and further replicated and validated the identified biomarker candidates in another cohort of patients (FIGS. 3 and 4). The levels of these newly identified biomarkers tested in the plasma significantly differed between the disease groups and healthy individuals.

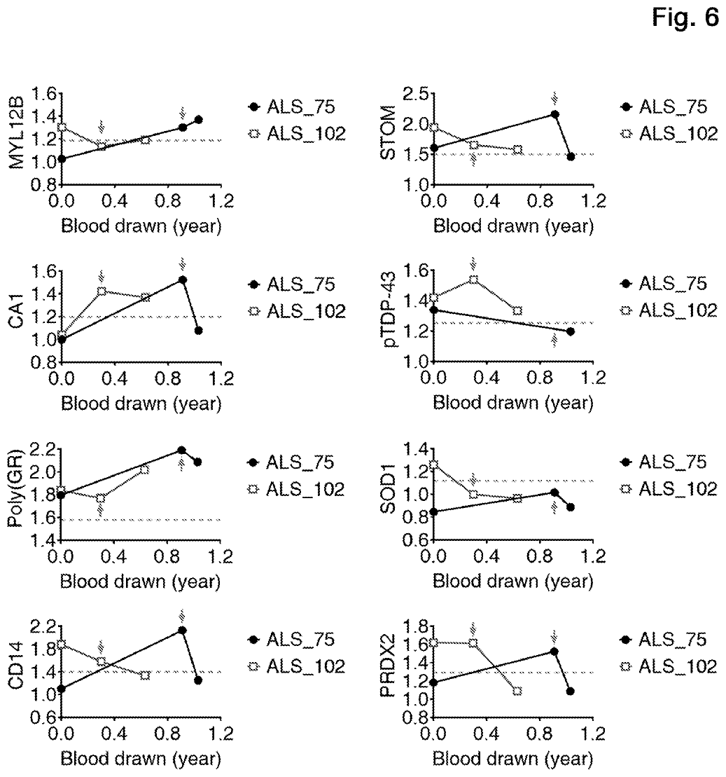

Edaravone, an antioxidant drug used in the clinic for ALS, reduces the plasma level of PRDX2 but not TDP-43 or GR repeat proteins, which reveals that the inefficacy of edaravone in clinical trials may be due to the inability to remove misfolded proteins and confirms a potential application of b-isox ELISA in the pharmacoresponse analysis of ALS (FIG. 6).

All of applicants' data suggested plasma b-isox-captured proteins can act as sensitive indicators of pathophysiological changes during the pathogenesis of neurodegenerative diseases and enable a precision medicine approach for neurodegenerative diseases.

### Methods for PD Diagnostic by Detecting CSF and Plasma b-isox-captured Proteins

Applicants developed a new chemical ELISA method, termed b-isox ELISA, for detecting the levels of specific proteins in b-isox precipitates (FIG. 2a).

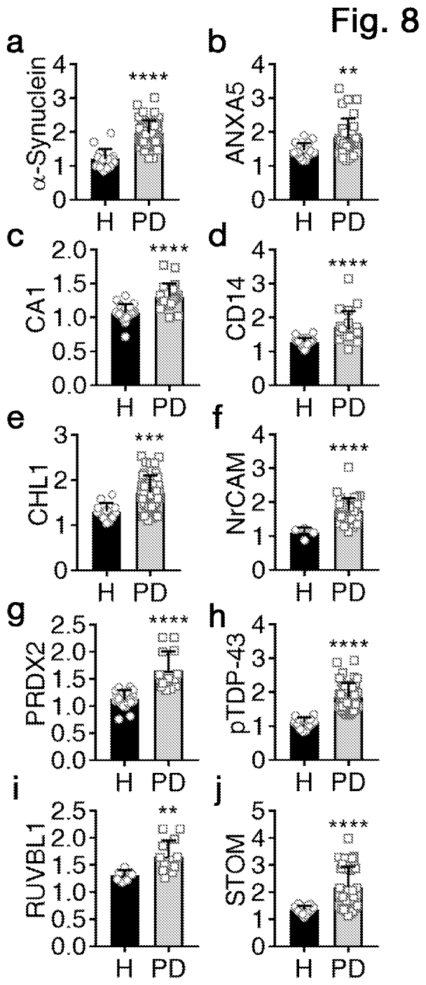

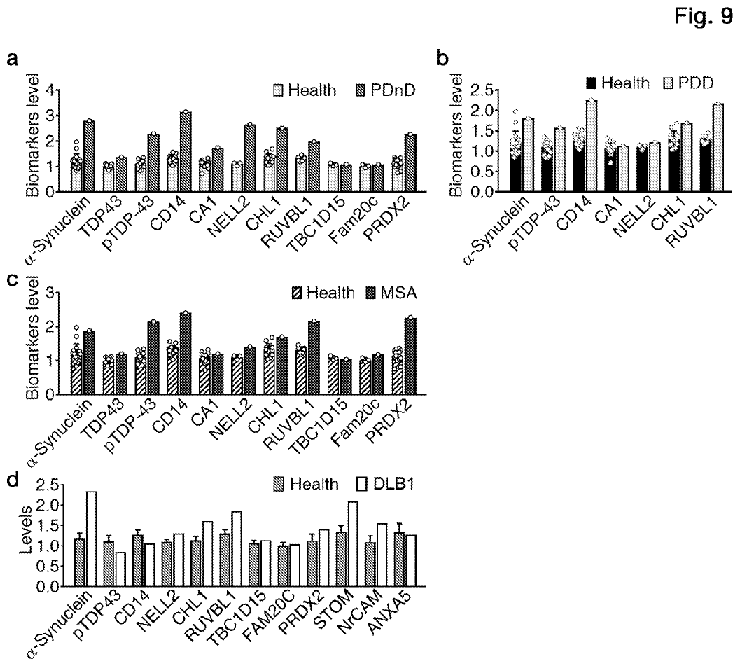

Applicants found the chemical ELISA detected the levels of specific b-isox captured proteins tested in plasma, and significantly differed between the neurodegenerative disease groups and HCs (FIGS. 8 and 9).

Applicants further identified novel biofluid biomarkers of PD by proteomics analysis of b-isox precipitates and further replicated and validated the identified biomarker candidates in another cohort of patients (FIGS. 8 and 9). The levels of these novel biomarkers tested in the plasma significantly differed between the disease groups and healthy individuals.

### Methods for AD Diagnostic by Detecting CSF and Plasma b-isox-captured Proteins

Applicants developed a new chemical ELISA method, termed b-isox ELISA, for detecting the levels of specific proteins in b-isox precipitates (FIG. 2a).

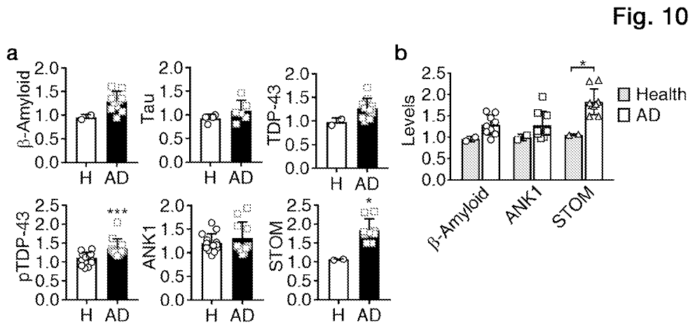

Applicants found the chemical ELISA detected the levels of specific b-isox captured proteins tested in plasma, and significantly differed between the neurodegenerative disease groups and HCs (FIG. 10).

Applicants further identified novel biofluid biomarkers of AD by proteomics analysis of b-isox precipitates and further replicated and validated the identified biomarker candidates in another cohort of patients (FIG. 10). The levels of these novel biomarkers tested in the plasma significantly differed between the disease groups and healthy individuals.

### Poly (GR) as a Novel Presymptomatic Biomarker for Inherited SOD1 and Sporadic ALS

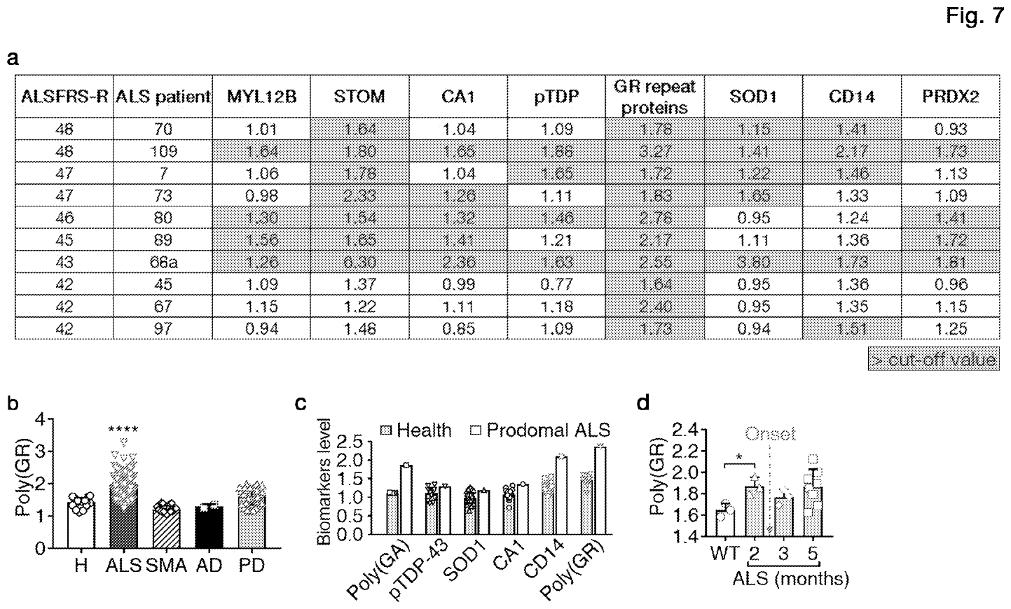

Aggregates deposition of dipeptide repeated proteins (poly GR, GP or GA) was a signature of C9orf72 inherited ALS. Unexpectedly, GR repeat proteins appeared at an early stage and constantly maintained a high level in the plasma of 87% ALS patients underlying disease progression of ALS (FIGS. 4, and 7a). These results suggested that poly (GR) proteins increase in the plasma of sporadic ALS, as inherited patients are only up to 10% of ALS. Our results shed light on a new pathophysiology role of plasma dipeptide repeated proteins (poly GR, GP or GA).

Increased level of poly (GR) does not be observed in SMA, AD and PD (FIG. 7b).

Applicants identified increased poly (GR) proteins appear in prodromal stage of inherited individual with C9orf72 mutations (FIG. 7c), and in the presymptomatic stage of SOD1 mice model (FIG. 7d). Thus, applicants suggested poly (GR) acts as a presymptomatic biomarker for C9orf72 ALS, sporadic ALS and SOD1 inherited ALS.

Additionally, applicants also found more 70% patients with increased poly (GA), and poly (GP) in their plasma. Applicants suggested poly (GA), and poly (GP) can be used as plasma biomarkers for not only C9orf72 inherited ALS, but also sporadic ALS diagnostic as well (FIG. 2).

### Description of Materials and Methods Used in the Examples

The following materials and methods were used in the Examples described below.

Reagents and Antibodies: b-isox was purchased from Sigma and Dalton dissolved in dimethyl sulfoxide (DMSO). Primary antibodies against SMN were purchased from BD Bioscience. The primary antibody against GFP was purchased from Roche. The primary antibodies against CA1 (#MBS1492724), and PRDX2 (#MBS7046127) were purchased from Mybiosource. The primary antibodies against MYL12B (#10324-1-AP), CD14 (#17000-1-AP), alpha-synuclein (#10842-1-AP), GR repeat proteins (#23978-1-AP), phospho-TDP-43 (Ser 409/410) (#22309-1-AP), PFN1 (#11608-1-AP), NrCAM (#14255-A-AP), STOM (#12046-1-AP), CHL1 (#25250-1-AP), NELL2 (#11268-1-AP), RUVBL1 (#10210-2-AP), ANXA5 (#11060-A-AP), and GLUT-1 (#21829-1-AP) were purchased from Proteintech. The primary antibodies against β-actin (#A1978) was purchased from Sigma. The primary antibodies against tau (#T9450) and ANK1 (#PA5-42203) were purchased from ThermoFisher. The primary antibodies against SOD1 (#A2770) was purchased from Cell signaling.

b-isox Precipitation: 10 mM biotinylated isoxazole was added to the human blood plasma or CSF to a final concentration of 100 to 200 μM. The mixtures were then incubated at 4° C. for 60 min, centrifuged at 15000 rpm for 15 min at 4° C., and the supernatant was discarded. The diameters of b-isox precipitates were measured.

b-isox precipitation and Enzyme-linked immunosorbent assay (ELISA) Blood samples from patients, healthy control were firstly collected through Blood Collection Tubes. Centrifugation of the tubes for 15 min at 2,200′ g. The resulting supernatant (upper layer) as the plasma sample. Before immunoassay, gently mixed 50-100 mL plasma (or CSF) with 0.5-1 mL b-isox through pipetting and rotated for 1 h at 4° C. Wash each streptavidin-coated microwell 3 times by 200 mL wash buffer (WB/(25 mM Tris, 150 mM NaCl; pH 7.2), 0.1% BSA, 0.05% Tween®-20) (do not allow wells to dry). Add 100 μL of the reaction mixtures to each well and incubate for 2 h with shaking (˜60 rpm) at room temperature (RT). Wash 3 times with 200 μL WB when reaction finished. Add 100 mL primary antibody diluent (appropriate primary antibody dilution in WB), incubating for 1 h at RT with shaking. At this step, a no primary antibody control should be included (Add antibody diluent alone in a sample well). Wash 3 times with 200 μL WB. Add 100 μL antibody diluent with appropriately diluted HRP-conjugated secondary antibody, incubating for 1 h at RT with shaking. Equilibrate the TMB substrate solution to RT at this step. Wash 3 times with 200 μL WB. Add 100 μL of the TMB Substrate Solution to each microplate well, incubating for 15-30 min until the color develops. Stop the reaction by adding 100 μL 2M sulfuric acid (or 2N HCl). Measure the optical density of at 450 nm by a microplate reader.

Depletion of plasma misfolded protein: Human plasma samples were incubated with A11 or B8H10 antibodies overnight at 4 degree with rotation (30 rpm). The 2nd day, reaction samples were incubated with 5 ul magnetic beads conjugated protein A/G beads (ThermoFisher) for 1 h at room temperature with rotation (30 rpm). Mixtures were then placed on a Magnet 3 min at RT to separate antibody-depleted plasma and antibody captured proteins. The supernatants were collected and further tested by b-isox ELISA with specific biomarkers.

### EXAMPLES

### Example 1: Analysis of the Quantities of b-isox-precipitates from the Plasma of Healthy Controls and Patients with ALS, AD, and PD

One milliliter of blood plasma from normal individuals and patients with ALS was incubated with b-isox, and centrifuged to pull down cross-β prion-like LC proteins (FIG. 1a). b-isox precipitates can be visually observed in samples from patients but not in samples from healthy people (FIG. 1a). The optimal condition of b-isox precipitation is the incubation of 100 ul plasma with b-iox for 60 min (FIG. 1b and c).

The statistical analysis of the size of b-isox precipitates from 100 μl plasma of normal subjects and patients with ALS, AD, and PD is shown in FIG. 1d. The accuracy of b-isox precipitation for ALS, AD, and PD is 98.4%, 81.8%, 92.8%, respectively.

Follow-up analysis of two patients with ALS showed that the size of b-isox precipitates was negatively correlated with the functional score (ALSFRS-R) (FIG. 1e). These results suggested that b-isox precipitates can be used not only in diagnostics but also for the prediction of future progression to ALS disability.

### Example 2: Differential Diagnosis of ALS by b-isox ELISA

To detect the levels of specific low-complexity (LC) proteins in b-isox precipitates, we developed a chemical ELISA method termed b-isox ELISA. A schematic diagram of b-isox ELISA is shown in FIG. 2a. Plasma was mixed with b-isox to generate b-isox-bound complexes, and then, we used streptavidin to capture b-isox-bound complexes, followed by traditional ELISA with a specific antibody against the targets of interest.

First, we analyzed the level of b-isox captured SOD1 in the plasma of control, ALS mice with the SOD1 G93A mutation and SMA mice by b-isox-based ELISA (FIG. 2b). We found that the increased level of SOD1 in precipitates isolated from the plasma of ALS mice is associated with the time period around the aggregation of misfolded SOD1 proteins in ALS mice. The level of SOD1 did not change in the plasma of SMA mice.

Consistently, we examined the SOD1 levels in the plasma of patients with an inherited SOD1 G93A mutation, and found the plasma level of SOD1 significantly increased in the inherited patient with ALSFRS-R 43 compared to controls (FIG. 2c).

Furthermore, by an analysis of ˜120 patients with ALS, the ˜10% of patients with high plasma SOD1 corresponds closely to the proportion of patients known from autopsy studies to have SOD1 inclusions in the spinal cord, as shown in FIG. 2d.

Next, we examined the p-TDP-43 levels in precipitates isolated from the plasma of patients with ALS by b-isox ELISA. Consistently, we found that the positivity ratio of patients with p-TDP-43 was correlated with the reported incidence rate of TDP-43 in ALS (FIG. 2e).

In a patient with high plasma level of p-TDP-43, we found that plasma levels of GR repeat proteins were elevated as well, but TDP-43 and SOD1 levels were not (FIG. 2f).

A longitudinal study revealed that both the plasma level of p-TDP-43 and the size of b-isox precipitates reduced during ALS progression (FIG. 2g).

We next examined the plasma levels of GR repeat proteins in normal controls and patients with ALS. The positivity ratio of patients with raised GR repeat proteins was up to ˜87.5%, which covers the group of sporadic ALS and has not be reported yet (FIG. 2h).

A representative profile of an ALS patient showed plasma p-TDP-43 and SOD1 proteins were not also elevated in a patient with high plasma levels of GR repeat proteins (FIG. 2i).

FIG. 2j shows a longitudinal tracking of the association of the plasma level of GR repeat proteins, the size of b-isox precipitates and ALSFRS-R.

### Example 3: Identifications and Validation of Novel Pathophysiological Biomarkers for ALS

By using proteomic analysis of b-isox-generated precipitates recovered from the CSF of patients with ALS, AD or PD, we discovered a few potential novel biomarkers of pathophysiology that involves in pathogenesis of ALS, AD or PD. The comprehensive flow chart of the experiment is shown in FIG. 3a. b-isox-captured precipitates from the plasma of healthy individuals and patients with ALS, AD or PD, were subjected to protein identification by LC-Ms/Ms, and differentially expressed proteins were categorized into membrane trafficking, exosome, metabolism, spliceosome, peptides and inhibitor, and chromosome and associated proteins according to the enriched gene ontology (GO) terms.

We replicated and validated the identified biomarker candidates in another cohort of patients and confirmed CA1, CD14, MYL12B, PRDX2, STOM, GLUT-1, SMN, β-Actin (ACTB), PFN1, poly (GA), and poly (GP), as biomarkers of pathophysiology in ALS (FIG. 3b). The levels of nine b-isox captured proteins tested in the plasma significantly differed between the disease groups and healthy individuals.

Unexpectedly, applicants found more 80% patients with increased dipeptide repeat proteins poly (GR), poly (GA), and poly (GP) in their plasma suggested these proteins can be used as biomarkers for not only C9orf72 inherited ALS, but also sporadic ALS diagnostic (FIGS. 2 and 3).

### Example 4: The Associations of the Plasma b-isox-captured Proteins and ALS Disability

To explore the relationship between proteinopathies of modulator/risk proteins and the progression to disability in patients with ALS, we analyzed the associations of the levels of pathogenic variants with the Amyotrophic Lateral Sclerosis Functional Rating Scale (ALSFRS-R) score. Plasma levels of pathogenic variants, including p-TDP-43, GR repeat proteins, MYL12B, STOM, SOD1, CD14, CA1, PRDX2, and ANXA5 were measured. Patients were subgrouped into four stages according to functional scores (FIG. 4a). ALSFRS-R was divided into four groups: stage I (score 40-48), II (score 30-39), III (score 20-29), IV (score 10-19). The percentage on the top represents the proportion of patients with increased biomarkers.

In addition to the appearance of high SOD1 in the early stage of ALS, a subgroup with low SOD1 appears at a later stage. Given that SOD1 inclusions have been detected in the motor neurons of sALS patients carrying the C9ORF72 repeat expansion, without SOD1 mutation, we deduced that inducing SOD1 misfolding is a common downstream event in ALS progression (FIG. 4a).

Additionally, PRDX2, MYL12B, and CD14 show similar trends, which drop-off after stage II (score 30-39) (FIG. 4b).

High GR repeat proteins maintain during progress to disability (FIG. 4a and c).

Notably, the appearance of high ANXA5 proteins in plasma of ALS patients only in the early stage of ALS reflects the occurrence of neuron turnover, which may trigger disease onset of ALS (FIG. 4d).

### Example 5: Longitudinal Tracking of b-isox-captured Proteins in the Plasma of ALS

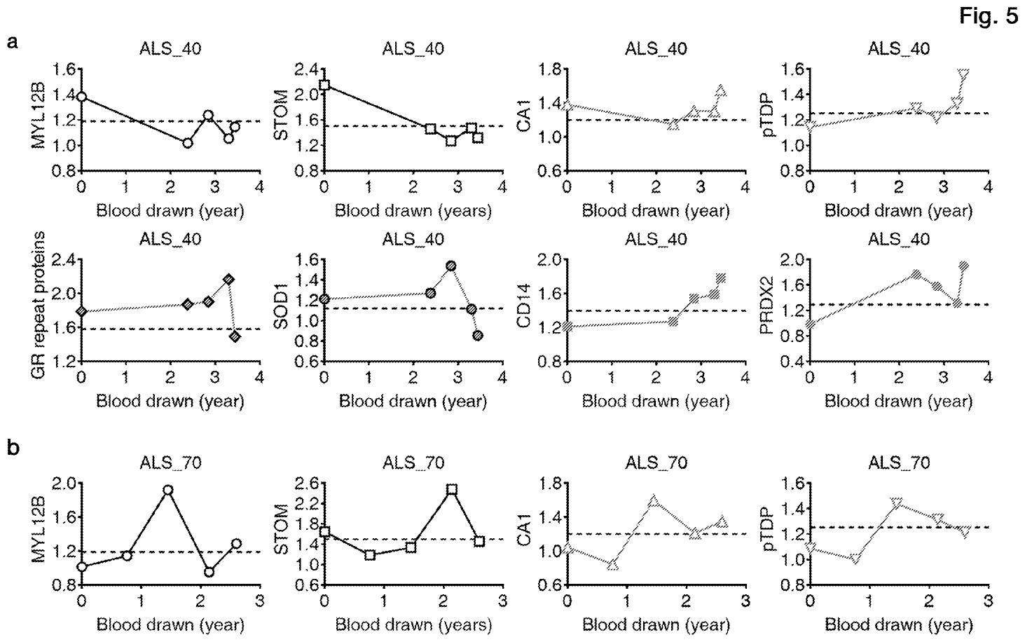

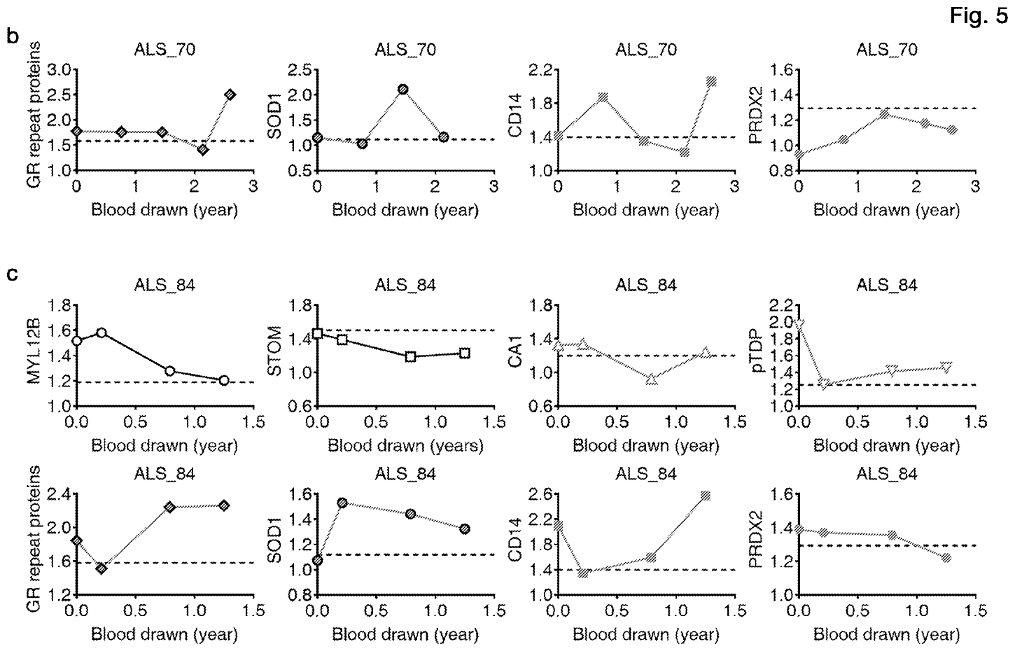

We performed b-isox ELISA analyses of progression to ALS in 23 participants and followed them with repeated clinical examinations up to 3.5 yr. We observed that b-isox-captured proteins in the plasma of patients dynamically change throughout the disease progression of ALS in an individual patient. Two examples were shown in FIG. 5.

### Example 6: Pharmacoresponse Analysis of Edaravone in ALS

By using b-isox ELISA to monitor the real-time pharmacoresponses of two ALS patients treated with edaravone, we found that edaravone treatments reduced plasma PRDX2 and CD14, but had no significant effects on plasma GR repeat proteins (FIG. 6). Edaravone is an anti-oxidant that may explain PRDX2 reduce. We deduced that the ineffectiveness of edaravone in ALS clinical trial may be due to fail on relief of pathological burden of misfolded GR repeat proteins.

### Example 7: Poly (GR) is a Very Early Biomarker for SOD1 Inherited and Sporadic ALS

Among all ALS biomarkers, which we obtained from the b-isox precipitation analysis, only GR repeat proteins appeared at an early stage and constantly maintained a high level in the plasma of patients underlying disease progression of ALS (FIGS. 4 and 7a). However, increased poly (GR) proteins are absent in SMA, AD and PD that suggested GR repeat proteins is a specific biomarker for ALS (FIG. 7b).

A single woman with inherited C9orf72 repeats at prodromal stage showed an increase of poly (GA), and CD14 and poly (GR) proteins, but no changes of p-TDP-43 and SOD1 (FIG. 7c).

Indeed, we observed the plasma GR repeat proteins increased at the presymptomatic stage and appeared earlier than plasma misfolded SOD1 in SOD1 G93A mice (FIG. 7d). These results suggested GR repeat proteins can be used as a very early biomarker for not only acting as prodromal biomarker of inherited C9orf72 ALS, but also involving in sporadic ALS and possible other types of inherited ALS, such as SOD1 and TDP-43 mutations.

### Example 8: Identification and Validation of Plasma Biomarkers of PD by b-isox ELISA

We analyzed patients with PD by b-isox ELISA, and the results showed there is a significant quantitative difference between the chemical precipitates of PD patients and healthy individuals. Using proteomic analysis, we comprehensively analyzed the precipitates isolated from CSF of patients with PD and obtained few PD biomarker candidates.

As expected, we confirmed the raised levels of synuclein, p-TDP-43, PRDX2,CHL1, NrCAM, STOM, CA1, CD14, ANXA5, NELL2, and RUVBL1 in the plasma of patients with PD by b-isox-ELISA (FIG. 8).

### Example 9: The Molecular Profiling of PD, PDD, MSA and DLB by b-isox ELISA of PD

The plasma from patients with PD, PDD, MSA and DLB were characterized by PD biomarkers (FIG. 9).

### Example 10: Identification and Validation of Plasma Biomarkers of AD by b-isox ELISA

We analyzed patients with AD by b-isox ELISA, and the results showed there is a significant quantitative difference between the chemical precipitates of AD patients and healthy individuals. Using proteomic analysis, we comprehensively analyzed the precipitates isolated from CSF of patients with AD.

As expected, we confirmed the raised levels of amyloid B, tau, p-TDP-43, TDP-43 ANK1 and STOM in the plasma of patients with AD by b-isox-ELISA (FIG. 10a).

Significantly, ANK1 and STOM show more sensitive than amyloid β by b-isox-ELISA (FIG. 10b).

### REFERENCES

1. Prof et al., Conformational disease, 1997, The Lancet, vol. 350, pp. 134-138.
2. Kiernan et al., Amyotrophic lateral sclerosis, 2017, The Lancet. Vol. 390, pp. 2084-2098.
3. Rosen et al., Mutations in Cu/Zn superoxide dismutase gene are associated with familial amyotrophic lateral sclerosis, 1993, Nature. Vol. 362(6415), pp. 59-62.
4. Neumann et al., Ubiquitinated TDP-43 in frontotemporal lobar degeneration and amyotrophic lateral sclerosis, 2006, Science, vol. 314, pp. 130-133.
5. Wang et al., TDP-43: an emerging new player in neurodegenerative diseases, 2008, Trends Mol. Med., vol. 14, pp.479-485.
6. DeJesus-Hernandez et al., Expanded GGGGCC hexanucleotide repeat in noncoding region of C9ORF72 causes chromosome 9p-linked FTD and ALS, 2011, Neuron. vol.72, pp.245-56.
7. Achmitz et al., Emerging Perspectives on Dipeptide Repeat Proteins in C9ORF72 ALS/FTD, 2021, The Lancet, vol. 397, pp. 1577-1590.
8. Scheltens et al., Alzheimer's disease, 2021, The Lancet, vol. 397, pp. 1577-1590.
9. Braak et al., Staging of brain pathology related to sporadic Parkinson's disease, 2003, Neurobiol. Aging, vol. 24(2), pp. 197-211.
10. Wang et al. The self-interaction of native TDP-43 C terminus inhibits its degradation and contributes to early proteinopathies, 2012, Nature Commun., Vol. 3, pp. 766.
11. Kato et al. Cell-free formation of RNA granules: low complexity sequence domains form dynamic fibers within hydrogels, 2012, Cell, vol. 149, pp. 753-767.
12. Han et al. Cell-free formation of RNA granules: bound RNAs identify features and components of cellular assemblies, 2012, Cell, vol. 149, pp. 768-779.
13. Brangwynne et al. Germline P granules are liquid droplets that localize by controlled dissolution/condensation, 2009, Science vol. 324, pp. 1729-1732.

Other embodiments of the invention will be apparent to those skilled in the art from consideration of the specification and practice of the invention disclosed herein. It is intended that the specification and examples be considered as exemplary only.

What is claimed is:

1. A method for detecting a human disease in a human subject, comprising,
optionally obtaining a biofluid sample from the subject,
adding an isoxazole to the obtained biofluid sample to form a biofluid isoxazole composition in the biofluid sample, and
detecting a presence of the biofluid isoxazole composition.

2. The method of claim 1, wherein the isoxazole is biotin-isoxazole, (6-(5-(Thiophen-2-yl)isoxazole-3-carboxamido)hexyl 5-((3aS,4S,6aR)-2-oxohexahydro-1H-thieno[3,4-d]imidazol-4-yl)pentanoate), or its salt or an analog thereof, especially biotin-isoxazole and very especially biotin-isoxazole.

3. The method of claims 1-2, wherein the biofluid sample is urine, whole blood, plasma, or serum, cerebrospinal fluid (CSF), saliva, or mucosa, such as, urine, saliva, CSF or plasma, and especially CSF or plasma.

4. The method of claims 1-3, wherein the human disease is Amyotrophic lateral sclerosis (ALS), Alzheimer's disease (AD), parkinson disease dementia (PDD), parkinson disease no dementia (PDnD), dementia with Lewy body (DLB), multiple system atrophy (MSA), spinal muscular atrophy (SMA), and limbic predominant age related TAR DNA-binding protein 43 (TDP-43) encephalopathy neuropathological change (LATE-NC), stroke, cerebral amyloid angiopathy (CAA), frontotemporal dementia (FTLD), diabetes, cancer, infectious disease, huntington disease, schizophrenia, aging-associated disease, or protienopathies, especially wherein the human disease is Amyotrophic lateral sclerosis (ALS), Alzheimer's disease (AD), parkinson disease dementia (PDD), parkinson disease no dementia (PDnD), dementia with Lewy body (DLB), multiple system atrophy (MSA), spinal muscular atrophy (SMA), and limbic predominant age related TAR DNA-binding protein 43 (TDP-43) encephalopathy neuropathological change (LATE-NC), stroke, cerebral amyloid angiopathy (CAA), or frontotemporal dementia (FTLD).

5. The method of claims 1-4, further comprising thereafter treating the human subject for the human disease or thereafter changing an existing treatment of the human subject for the human disease based on the detecting the presents of the biofluid isoxazole composition, such as a treatment including pharmaceutical therapy for the human disease, and optionally further comprising diagnosing the human disease in the human subject.

6. The method of claims 1-5, wherein the concentration of isoxazole ranges from 0.075 mM to 0.225 mM, preferably from 0.100 mM to 0.200 mM, in the biofluid sample.

7. The method of claims 1-6, wherein the biofluid isoxazole composition is in a precipitate.

8. The method of claim 7, wherein the human disease is Amyotrophic lateral sclerosis (ALS), Alzheimer's disease (AD), parkinson disease dementia (PDD), parkinson disease no dementia (PDnD), dementia with Lewy body (DLB), multiple system atrophy (MSA), spinal muscular atrophy (SMA), and limbic predominant age related TAR DNA-binding protein 43 (TDP-43) encephalopathy neuropathological change (LATE-NC), stroke, cerebral amyloid angiopathy (CAA), or frontotemporal dementia (FTLD).

9. The method of claim 8, wherein the human disease is ALS, AD, DLB, MSA or PDD or PDnD.

10. The method of claim 9, wherein the human disease is ALS, such as sporadic ALS.

11. The method of claims 7-10, further comprising monitoring the size of the precipitate to monitor the progression of ALS in the subject.

12. The method of claims 1-6, further comprising adding a polypeptide to biofluid isoxazole composition in the biofluid sample to facilitate detection by an immune assay, such as a blot assay, a chemiluminescence immunoassay, an enzyme-linked immunosorbent assay (ELISA), a light scattering immunoassay, a radiolabeled immunoassay, in particular, ELISA or a Western blot.

13. The method of claim 12, wherein the human conformational disease is Amyotrophic lateral sclerosis (ALS), Alzheimer's disease (AD), parkinson disease dementia (PDD), parkinson disease no dementia (PDnD), dementia with Lewy body (DLB), multiple system atrophy (MSA), spinal muscular atrophy (SMA), and limbic predominant age related TAR DNA-binding protein 43 (TDP-43) encephalopathy neuropathological change (LATE-NC), stroke, cerebral amyloid angiopathy (CAA), or frontotemporal dementia (FTLD).

14. The method of claims 12-13, wherein the polypeptide is (a) an antibody, or (b) an immunoglobulin chain, or a binding domain thereof which binds to the biofluid isoxazole composition.

15. The method of claim 14, wherein the human diseases is ALS, and wherein the polypeptide for detecting ALS is an antibody against SOD1, C9orf72 dipeptide repeats, PFN1, PRDX2, phospho-TDP-43, CA1, MYL12B, CD14, ANXA5, STOM, SMN, ACTB, or GLUT1, such as SOD1, MYL12B, CD14 and p-TDP-43, and especially SOD1 and p-TDP-43.

16. The method of claim 14, wherein the human diseases is AD, and wherein polypeptide for detecting AD is an antibody against APP, phospho-TDP-43, TDP-43, Tau, STOM, or ANK1.

17. The method of claim 14, wherein the human diseases is PD, and wherein polypeptide for detecting PD is an antibody against synuclein, CHL1, NELL2, p-TDP-43, NrCAM, ANK1, STOM, PRDX2, CA1, CD14, and RUVBL1.

18. The method of claims 15-17, wherein biofluid is plasma or CSF.

19. The method of claims 12-18, wherein the method is ELISA, such as direct, indirect, sandwich, or competitive ELISA.

20. Use of an isoxazole to detect a human disease in any method of claims 1-19.

21. A method for detecting a human disease in a human subject, comprising,
detecting a presence of a biomarker from an obtained biofluid sample, and
optionally obtaining a biofluid sample from the subject to obtain the obtained biofluid sample, and
wherein the human disease is selected from sporadic Amyotrophic lateral sclerosis (ALS), Alzheimer's disease (AD), parkinson disease dementia (PDD), and parkinson disease no dementia (PDnD),
wherein, for sporadic ALS, the biomarker is selected from C9orf72 dipeptide repeats, PRDX2, CA1, MYL12B, CD14, ANXA5, STOM, SMN, and GLUT1;
wherein, for AD, the biomarker is selected from STOM and ANK1, and
wherein for DLB, MSA, PDD or PDnD, the biomarker is selected from CHL1, NELL2, NrCAM, ANK1, STOM, PRDX2, CA1, CD14, and RUVBL1.

22. The method of claim 21, wherein the obtained biofluid sample is urine, whole blood, plasma, or serum, cerebrospinal fluid (CSF), saliva, or mucosa, such as, urine, saliva, CSF or plasma, and especially CSF or plasma.

23. The method of claims 21-22, wherein the human disease is ALS.

24. The method of claims 21-22, wherein the human disease is AD.

25. The method of claims 21-22, wherein the human disease is DLB, MSA, PDD or PDnD.

26. The method of claims 21-25, further comprising thereafter treating the human subject for the human disease or thereafter changing an existing treatment of the human subject for the human disease based on the detecting the presents of the biofluid isoxazole composition, such as a treatment including pharmaceutical therapy for the human disease, and optionally further comprising diagnosing the human disease in the human subject.

27. The method of claims 21-26, further comprising adding a polypeptide to the obtained biofluid sample to facilitate detection by an immune assay, such as a blot assay, a chemiluminescence immunoassay, an enzyme-linked immunosorbent assay (ELISA), a light scattering immunoassay, a radiolabeled immunoassay, in particular, ELISA or a Western blot.

28. The method of claim 27, wherein the polypeptide is (a) an antibody, or (b) an immunoglobulin chain, or a binding domain thereof which binds to the biomarker.

29. The method of claim 28, wherein the human diseases is sporadic ALS, and wherein the polypeptide for detecting sporadic ALS is an antibody against C9orf72 dipeptide repeats, PRDX2, CA1, MYL12B, CD14, ANXA5, STOM, SMN, or GLUT1, such as C9orf72 dipeptide repeats.

30. The method of claim 28, wherein the human diseases is AD, and wherein polypeptide for detecting AD is an antibody against STOM or ANK1.

31. The method of claim 28, wherein the human diseases is DLB, MSA, PDD or PDnD, and wherein polypeptide for detecting PD is an antibody against CHL1, NrCAM, ANK1, STOM, PRDX2, CA1, CD14, or RUVBL1.

32. The method of claims 29-31, wherein biofluid is plasma or CSF.

33. The method of claims 27-32, wherein the method is ELISA, such as direct, indirect, sandwich, or competitive ELISA.

34. Use of the biomarker to detect the human disease in any method of claims 21-33.

35. A method for detecting Amyotrophic lateral sclerosis (ALS) in a human subject, comprising,
detecting a presence of a dipeptide repeat protein from an obtained biofluid sample, and
optionally obtaining a biofluid sample from the subject to obtain the obtained biofluid sample.

36. The method of claim 35, wherein the biofluid sample is urine, whole blood, plasma, or serum, cerebrospinal fluid (CSF), saliva, or mucosa, such as, urine, saliva, CSF or plasma, and especially CSF or plasma.

37. The method of claims 35-36, wherein the dipeptide repeat protein is selected from poly (GR), poly (GP), and poly (GA).

38. The method of claims 35-37, further comprising thereafter treating the human subject for the human disease or thereafter changing an existing treatment of the human subject for the human disease based on the detecting the presents of the biofluid isoxazole composition, such as a treatment including pharmaceutical therapy for the human disease, and optionally further comprising diagnosing the human disease in the human subject.

39. The method of claims 35-38, further comprising adding a polypeptide to the obtained biofluid sample to facilitate detection by an immune assay, such as a blot assay, a chemiluminescence immunoassay, an enzyme-linked immunosorbent assay (ELISA), a light scattering immunoassay, a radiolabeled immunoassay, in particular, ELISA or a Western blot.

40. The method of claim 39, wherein the polypeptide is (a) an antibody, or (b) an immunoglobulin chain, or a binding domain thereof which binds to the biomarker.

41. The method of claim 40, wherein the human diseases is sporadic ALS, and wherein the polypeptide for detecting sporadic ALS is an antibody against dipeptide repeat proteins, PRDX2, CA1, MYL12B, CD14, ANXA5, STOM, SMN, or GLUT1, such as C9orf72 dipeptide repeats.

42. The method of claims 39-41, wherein biofluid is plasma or CSF.

43. The method of claims 39-42, wherein the method is ELISA, such as direct, indirect, sandwich, or competitive ELISA.

44. Use of the dipeptide repeat protein to detect sporadic ALS in any method of claims 35-43.

45. A method for detecting sporadic or SOD1 inherited Amyotrophic lateral sclerosis (ALS) in a human subject at the presymptomatic and prodromal stage, comprising,
detecting a presence of a dipeptide repeat protein from an obtained biofluid sample, and
optionally obtaining a biofluid sample from the subject to obtain the obtained biofluid sample.

46. The method of claim 45, wherein the biofluid sample is urine, whole blood, plasma, or serum, cerebrospinal fluid (CSF), saliva, or mucosa, such as, urine, saliva, CSF or plasma, and especially CSF or plasma.

47. The method of claims 45-46, wherein the dipeptide repeat protein is poly (GR).

48. The method of claims 45-47, further comprising thereafter treating the human subject for the human disease or thereafter changing an existing treatment of the human subject for the human disease based on the detecting the presents of the biofluid isoxazole composition, such as a treatment including pharmaceutical therapy for the human disease, and optionally further comprising diagnosing the human disease in the human subject.

49. The method of claims 45-48, further comprising adding a polypeptide to the obtained biofluid sample to facilitate detection by an immune assay, such as a blot assay, a chemiluminescence immunoassay, an enzyme-linked immunosorbent assay (ELISA), a light scattering immunoassay, a radiolabeled immunoassay, in particular, ELISA or a Western blot.

50. The method of claim 49, wherein the polypeptide is (a) an antibody, or (b) an immunoglobulin chain, or a binding domain thereof which binds to the biomarker.

51. The method of claim 50, wherein the polypeptide for detecting sporadic or SOD1 inherited ALS is an antibody against dipeptide repeat proteins, PRDX2, CA1, MYL12B, CD14, ANXA5, STOM, SMN, or GLUT1, such as C9orf72 dipeptide repeats.

52. The method of claims 49-51, wherein biofluid is plasma or CSF.

53. The method of claims 49-52, wherein the method is ELISA, such as direct, indirect, sandwich, or competitive ELISA.

54. Use of the dipeptide repeat protein to detect sporadic or SOD1 inherited ALS in any method of claims 45-53.

55. Use of any described substance or composition for diagnosing the human disease in each claim 1-53.
